(19) United States
(12) Patent Application Publication
(10) Pub. No.: US 2015/0223833 A1
(43) Pub. Date: Aug. 13, 2015

(54) TROCAR SEAL ASSEMBLY

(71) Applicants: Jared COFFEEN, Hollister, CA (US); Polina A. SEGALOVA, Redwood City, CA (US)

(72) Inventors: Jared COFFEEN, Hollister, CA (US); Polina A. SEGALOVA, Redwood City, CA (US)

(21) Appl. No.: 14/615,773

(22) Filed: Feb. 6, 2015

Related U.S. Application Data
(60) Provisional application No. 61/939,459, filed on Feb. 13, 2014.

(51) Int. Cl. A61B 17/34; A61B 17/02

(52) U.S. Cl. CPC A61B 17/3423; A61B 17/0218; A61B 17/3498; A61B 2017/00955

(57) ABSTRACT

A cannula and trocar assembly, including a septum seal assembly is provided. The septum seal assembly includes a shield and a septum seal for creating a gas-tight seal around a surgical instrument that is inserted into and through the cannula assembly without causing damage to the seal assembly.

### CROSS-REFERENCE TO RELATED APPLICATIONS

This claims the benefit of U.S. Provisional Application No. 61/939,459, filed Feb. 13, 2014.

### BACKGROUND OF THE INVENTION

The present invention generally relates to medical apparatus seal assemblies, and more particularly to medical and surgical trocar seal assemblies.

Laparoscopy is a common surgical procedure which uses one or more small incisions and involves carbon dioxide gas being pumped into a patient to expand the abdomen, the process of which is referred to as insufflation. Insufflation allows the surgeon a better view of the internal organs and other internal body parts. A laparoscope is inserted through an incision to look at the internal organs, often times during the surgical procedure, to allow the surgeon to have a precise view of the surgical space.

It is necessary to retain as much insufflation gas in the abdominal cavity as possible and to maintain a constant gas pressure, while allowing access by instruments. Thus the seal(s) must allow easy access of the instruments through the cannula while retaining as much insufflation gas in the surgical space as reasonably possible in order to maintain the insufflation gas pressure.

To achieve this function, many different designs of seals have been employed. However, the seal assembly must not only provide a gas-tight or nearly gas-tight seal around an instrument when the instrument is extended into the cannula and into the surgical space, but also preferably allow for some lateral movement or angled movement of the instrument, allow easy insertion and withdrawal of the instrument without damaging the seal, and also employ protection against being damaged by a sharp instrument as it is inserted through the seal assembly.

The trocar assembly and cannula seal assembly disclosed herein perform all of these functions, while being cost effective to manufacture. One embodiment of the inventive trocar assembly comprises a housing with an opening and a unitary monolithic septum seal having an outer projection attached to the housing. The septum seal has an outer seal wall depending from the outer projection, and an inner seal wall connected to the outer seal wall and positioned inwardly with respect to the outer seal wall, defining a space between the outer seal wall and the inner seal wall. A sealing member extends inwardly from the inner seal wall and has an aperture substantially aligned with the housing opening. The inner seal wall and the sealing member together define an interior seal space. A shield for the protection of the septum seal from being perforated by a surgical instrument resides at least partially within the interior seal space and is expandable to allow different sized instruments to pass through it.

Another embodiment is a trocar assembly comprising a cannula assembly having a distal tubular portion and a proximal hub portion, a valve at least partially disposed within the cannula proximal hub portion, the valve having an opening to allow entry of an instrument into the distal tubular portion of the cannula, and a seal housing attached to the cannula proximal hub portion. The seal housing has a proximal opening sized to allow a surgical instrument therethrough and has at least one wall defining a seal housing interior. The trocar assembly of this embodiment also includes a unitary monolithic septum seal at least partially disposed within the seal housing interior and which has an outer member attached to the seal housing. The seal has a support member which is attached to the outer member of the septum seal, and a sealing portion is attached to the support member. The sealing portion is substantially cylindrically shaped and has a first end that is open and a second end that is substantially closed by a sealing member which has an aperture therein for receiving an instrument.

Yet another embodiment is a universal seal assembly for a trocar which includes a housing having an interior and a seal member at least partially disposed within the interior of the housing. The seal member has a proximal lip extending annularly and is engaged with the housing. The lip comprises an inner circumferential portion. A first seal wall extends distally from the inner circumferential portion of the proximal lip and has a distal edge. A second seal wall extends from the distal edge of the first seal wall, with the first seal wall and the second seal wall together defining an outer support member. A third seal wall extends from the second seal wall and a sealing member extends inwardly from the third seal wall, the sealing member having an aperture in it. The third seal wall and the sealing member together define an interior seal space, and the first seal wall, the second seal wall, and the third seal wall are positioned with respect to one another to create an s-shaped structure for allowance for off-axis movement of instruments received by the seal member.

Other advantages, objects and/or purposes of the invention will be apparent to persons familiar with constructions of this general type upon reading the following specification and inspecting the accompanying drawings.

### BRIEF DESCRIPTION OF THE DRAWINGS

FIG. 1 is a perspective view of a laparoscopic trocar assembly including a cannula assembly, an optical obturator assembly, and a laparoscope.

FIG. 2 is a perspective view of the cannula assembly of FIG. 1 with the seal housing removed from the remainder of the cannula assembly.

FIG. 3 is a bottom plan view of the cannula assembly of FIG. 1.

FIG. 4 is a cross-sectional view of the cannula assembly of FIG. 1, taken along lines IV-IV in FIG. 3.

FIG. 5 is a perspective exploded view of the seal housing, seal assembly, and retainer ring of the cannula assembly of FIG. 1.

FIG. 6 is a perspective exploded view of the seal assembly of FIG. 5.

FIG. 7 is a top plan view of the septum seal of the seal assembly of FIG. 5.

FIG. 8 is a cross-sectional view of the septum seal of FIG. 7, taken along the lines VIII-VIII in FIG. 7.

FIG. 9 is an enlarged section view of the proximal outer lip of the septum seal of FIG. 8.

FIG. 10 is a top plan of the shield of the seal assembly of FIG. 6.

FIG. 11 is a cross-sectional view of the shield of FIG. 10, taken along the line XI-XI in FIG. 10.

FIG. 12 is a perspective view of a second embodiment of a shield of the seal assembly.

FIG. 13 is a cross-sectional view of the shield of FIG. 12, taken along line XIII-XIII in FIG. 12.

FIG. 14 is a perspective view of a third embodiment of a shield of the seal assembly.

FIG. 15 is a cross-sectional view of the shield of FIG. 14, taken along line XV-XV in FIG. 14.

FIG. 16 is a perspective view of a fourth embodiment of a shield of the seal assembly.

FIG. 17 is a cross-sectional view of the shield of FIG. 16, taken along line XVII-XVII in FIG. 16.

### DETAILED DESCRIPTION OF THE PREFERRED EMBODIMENTS

Certain terminology will be used in this description for convenience and reference only, and will not be limiting. For example, the words “upwardly,” “downwardly,” “rightwardly,” and “leftwardly” will refer to directions in the drawings to which reference is made. The words “inwardly” and “outwardly” will refer to directions toward and away from, respectively, the geometric center of the arrangement and designated parts thereof. The words “forwardly” and “distally” will refer to the direction toward the end of the arrangement which is closest to the patient, and the words “rearwardly” and “proximally” will refer to the direction toward the end of the arrangement which is furthest from the patient. This terminology will include the words specifically mentioned, derivatives thereof, and words of similar import.

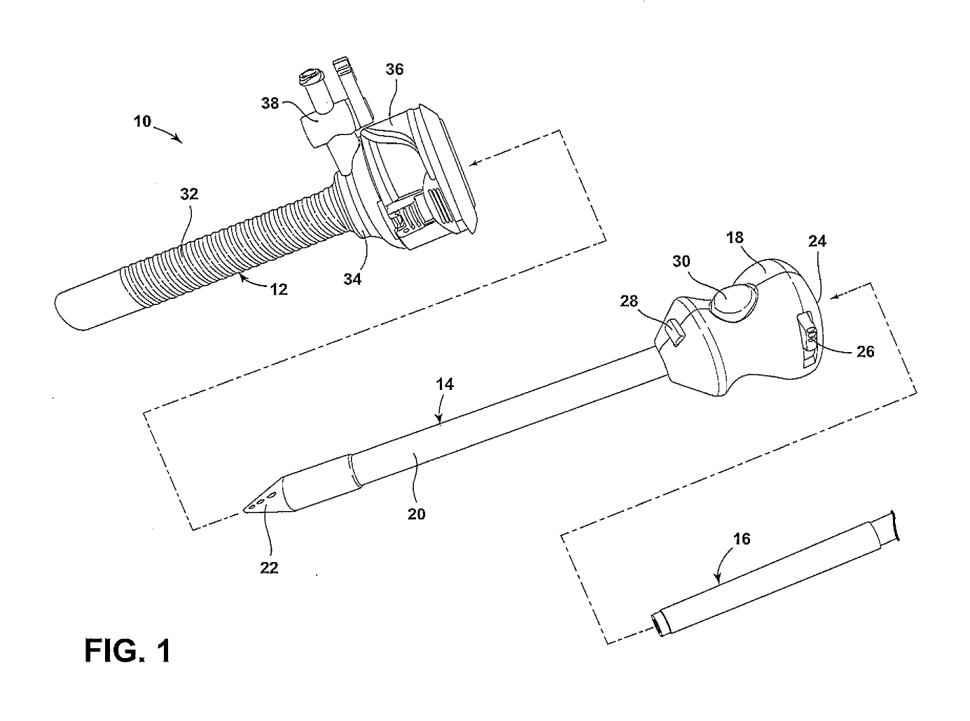

The preferred embodiment is a laparascopic trocar assembly 10 that includes a cannula assembly 12, an obturator assembly 14, and a laparoscope 16, as shown in FIG. 1.

The obturator assembly 14 preferably includes an obturator hub 18 which is connected to a tubular shaft 20 which has an optically clear tip 22 attached at its distal end. The shaft 20 is hollow, and made of a rigid metal, although it is contemplated that the shaft 20 could be made of other materials, and the hub 18 has an opening 24 which opens to a passageway through the hub 18 and which communicates with the interior of the shaft 20.

Accordingly, the laparoscope 16 may be inserted through the opening 24, through the interior of the hub 18, through the shaft 20, and into the optically clear tip 22. The hub includes a lock assembly 26 for locking the laparoscope 16 in place with respect to the obturator assembly 14 during use, and an engagement and disengagement system that includes hooks 28 which may engage with openings or grooves in a housing of the cannula assembly 12 to secure the obturator assembly 14 to the cannula assembly 12. Pads 30 may be moved by pressing them inwardly to disengage the hooks 28 from the housing of the cannula assembly 12.

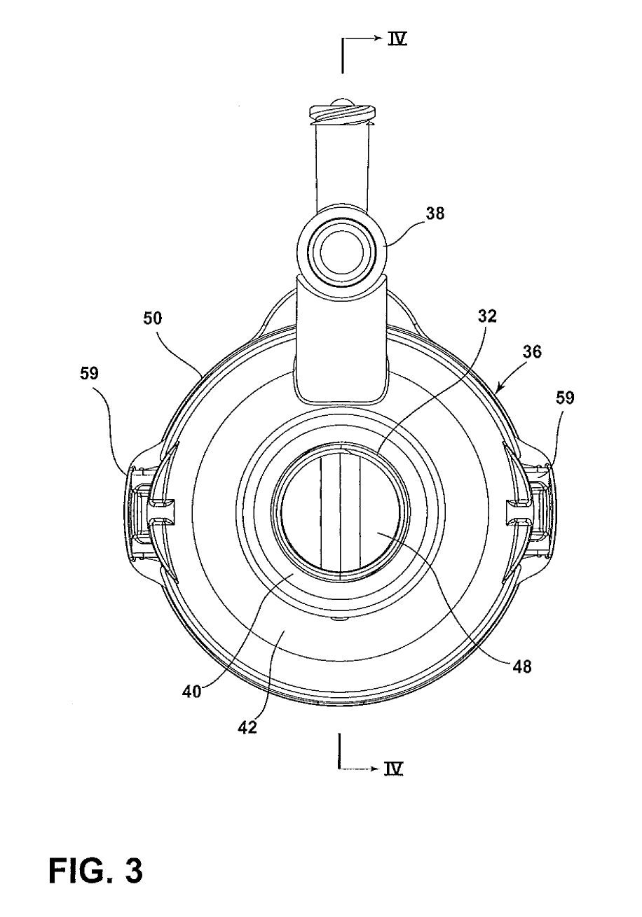

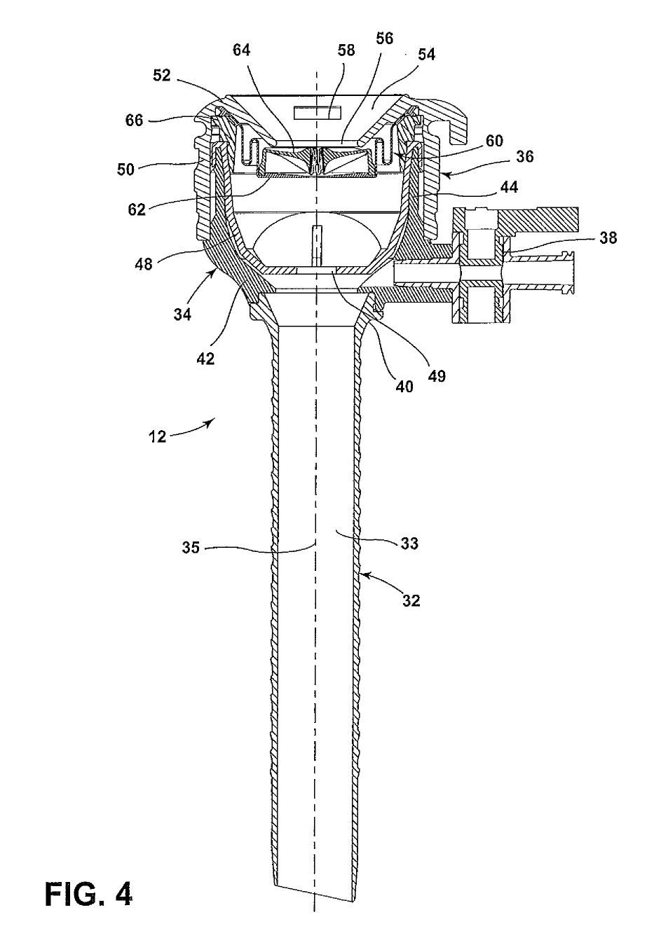

As shown in FIGS. 1-4, the cannula assembly generally includes a cannula 32, a hub 34, a seal housing 36, and a gas valve 38. The cannula 32 has a hollow interior 33 and extends along a longitudinal axis 35 (see FIG. 4).

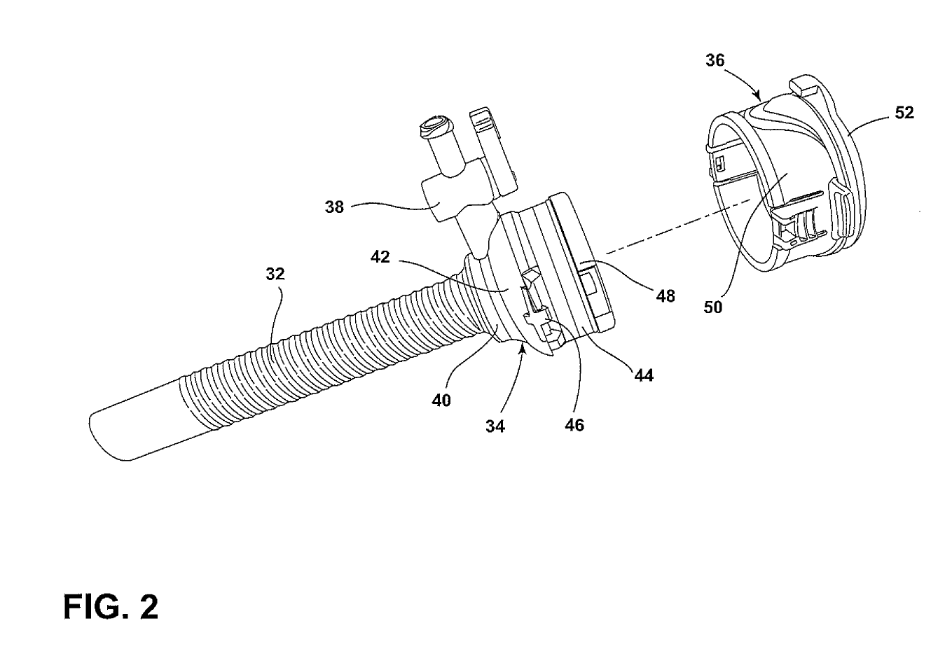

Best seen in FIG. 4, the hub 34 is fixedly attached to the cannula 32 at an annular base 40 at the proximal end of the cannula 32. The hub 34 includes a distal frustoconical section 42 and a cylindrical section 44 extending proximally from the frustoconical section 42. The outer diameter of the cylindrical section 44 is less than the diameter of the proximal end of the frustoconical section 42 such that the housing 36 may be slid over the cylindrical section 44 to engage with the frustoconical section 42. A portion of the valve 38, which is preferably in the form of a stopcock but may be other configurations, is attached to the frustoconical section 42 and is preferably integrally formed therewith. Extending proximally from the outer edge of the frustoconical section 42 are two opposing hooks 46 (see FIG. 2) for engagement with the housing 36, which is described in more detail below. At least partially inside the hub 34 resides a lower seal 48, preferably in the form of a duckbill valve. The lower seal 48 may be fixedly attached or removable from the hub 34. The lower seal has a central opening 49, which in the case of a duckbill valve, is a slit.

As shown best in FIGS. 2 and 4, the seal housing has a substantially cylindrical outer wall 50 which depends from a cap 52. The cap 52 is preferably affixed to, and may be integrally formed with, the cylindrical wall 50. The cap 52 includes a central frustoconical-shaped port 54 for receiving a surgical instrument and urging the instrument toward the longitudinal axis 35 during entry of the instrument into the housing 36. The port 54 includes a central opening 56 which allows entry of various-sized instruments into the housing 36 for entry into and through the cannula 32. The port 54 of the cap 52 also includes a plurality of apertures 58, in the form of slots, for receiving the engagement hooks 28 of the obturator assembly 14.

As part of the cylindrical wall 50 of the seal housing 36, opposing latches 59 depend from an upper portion of the cylindrical wall 50. Each latch 59 is slightly movable, but remains biased toward its initial position as part of the cylindrical wall 50. Thus, when the housing 36 is moved over the cylindrical section 44 and into engagement with the frustoconical section 42 of the hub, the latches 59 engage with hooks 46 and are moved inwardly with respect to the rest of the cylindrical wall 50. Because the latches 59 are created in such a manner to be biased toward their initial position, after passing a bulge part of the hooks 46, each latch 59 moves slightly outwardly to engage a hook 46 and hold the housing in place with respect to the hub 34. Pressing the latches 59 inwardly with respect to the remainder of the cylindrical wall 50 will move the latches past the innermost portion of bulge parts of the hooks 46 to allow removal of the seal housing 36 from the cannula hub 34.

At least partially within, and preferably entirely within, the housing 36 is an upper seal assembly 60. The upper seal assembly 60 generally includes a septum seal 62 and a shield 64. The upper seal assembly 60 is positioned proximally with respect to the opening 49 in the lower seal 48.

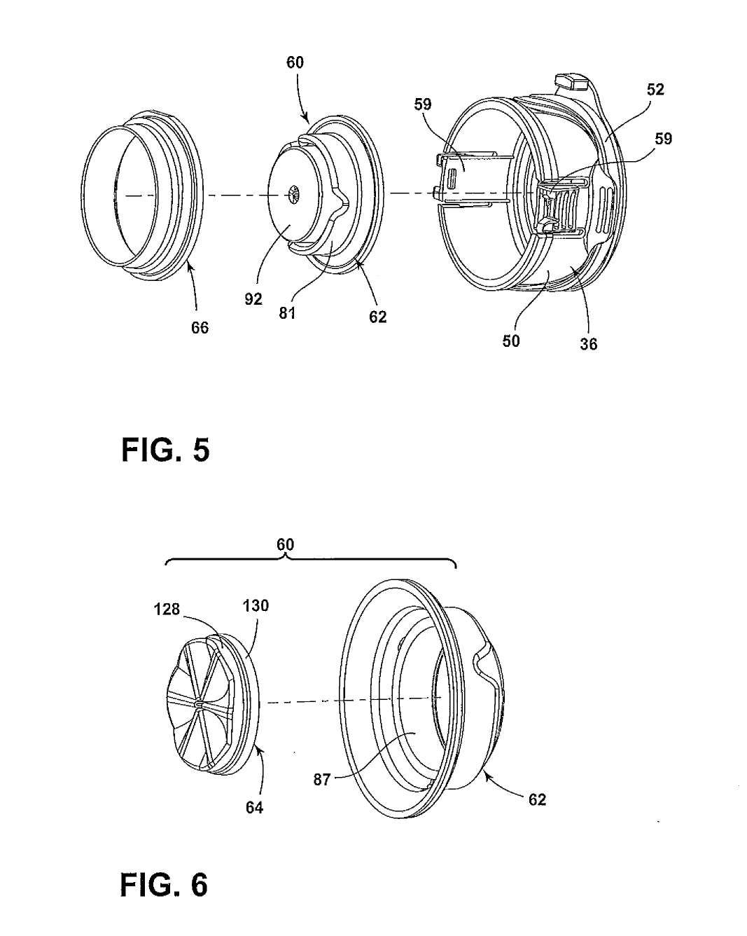

The shield 64 is seated within a portion of the septum seal 62 (see FIG. 4). A seal retainer ring 66 engages with an inner portion of the housing 36 to hold the septum seal 62 and shield 64 in place with respect to the housing 36. Adhesive or other means may be used to assist in affixing the upper seal assembly 60 to the housing 36. As shown in FIGS. 5-6, the shield 64 is inserted into the septum seal 62, which is in turn inserted and attached to the housing 36. The seal retainer ring 66 is then inserted and attached to the housing to retain the upper seal assembly 60 in the housing 36.

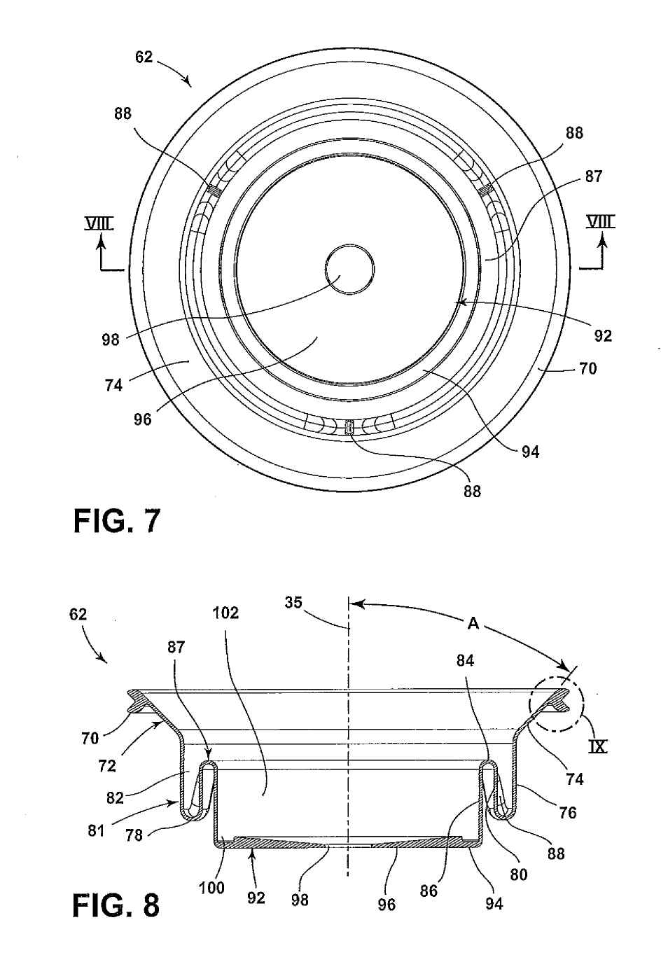

The septum seal 62 is shown in detail in FIGS. 7-9. At its proximal end the septum seal 62 has an outwardly radially positioned outer lip 70, which is described in more detail below. The outer lip 70 engages with the interior of the seal housing 36, as shown in FIG. 4. A first seal wall 72, in two portions, depends from the outer lip 70. The first seal wall 72 includes an upper angled portion 74, which defines a frustoconical portion, extending inwardly toward the longitudinal axis 35 as it extends downwardly from the outer lip 70, as it is oriented in FIG. 8. The upper angled portion 74 is disposed at an angle A with respect to the longitudinal axis 35, as shown in FIG. 8. Angle A is preferably between 40° and 50°, and more preferably about 45°. Depending from the upper angled portion 74 is a lower first wall portion 76. The lower first wall portion 76 extends substantially parallel to the longitudinal axis 35 and thus is substantially cylindrical in nature. From the bottom of the lower first wall portion 76 extends a u-shaped bottom 78.

Extending upwardly from the bottom 78 is a second seal wall 80, which is substantially parallel to the longitudinal axis 35. A space 82 exists between the first wall portion 76 and the second seal wall 80. The spacing and positioning of the first wall portion 76, the bottom 78, and the second seal wall 80 create a support member 81 which assists in supporting the structure of the septum seal 62 and assists in biasing an instrument inserted through the septum seal 62 toward the longitudinal axis 35. Extending from the second wall 80 is a u-shaped upper portion 84, and extending downwardly from the upper portion 84 is a third wall 86. The third wall 86 is positioned inwardly with respect to second wall 80, and the combination of the second wall 80, the upper portion 84, and the third wall 86 results in an inner ring 87, which is spaced inwardly with respect to the first wall lower portion 76. The walls 76, 80, 86 upper portion 84, and bottom 78 together create an s-shaped structure which allows for off-axis entry and movement of instruments.

To assist in stability, one or more ribs 88 are preferably used. Each rib 88 is attached to both the first wall lower portion 76 and the second wall 80, and sits within the space 82 between those two walls. In a preferred embodiment, three equally spaced ribs are used, with each rib 88 being spaced 120° from another rib. However, more or less ribs may be used and/or different spacings may be employed.

Extending inwardly from the third wall 86 is a sealing member 92. The sealing member is substantially perpendicular to the longitudinal axis 35 and includes an outer narrow portion 94, an inner ramp portion 96, and an aperture 98. The outer narrow portion 94 preferably extends about the entire circumferential distance of the sealing member 92 and extends from the third wall 86 inwardly to the inner ramp portion 96, creating an annular groove 100. The annular groove 100 is sized and shaped to receive a portion of the shield 64. The structure of the lower portion of the septum seal 62 results in the third wall 86 and the sealing member 92 creating a substantially cylindrical space 102 with an upper open portion and a substantially closed bottom portion defined by the sealing member 92.

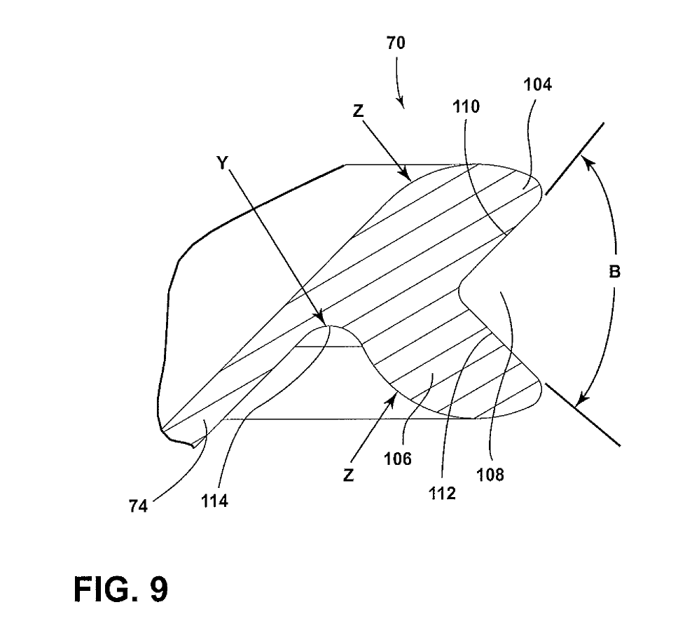

The outer lip 70 of the septum seal 62 has a fin shape with an upper member 104 and a lower member 106, as shown in FIG. 9. A v-shaped notch 108 extends inwardly to define the upper member 104 and lower member 106, and preferably has two substantially flat surfaces 110, 112. The surfaces 110 and 112 are at an angle B with respect to one another, as shown in FIG. 9. Angle B may be any angle which results in a useful outer lip, but is preferably between 80° and 100°, and more preferably is about 90°. The outer lip 70 extends outwardly from the upper angled portion 74 of the first seal wall. A curved portion 114 attaches the upper angled portion 74 of the first seal wall 72 to the outer lip 70. The radius of the curved portion 114 is designated as Y in FIG. 9 and is sized and oriented such that the outer lip extends substantially perpendicularly to the longitudinal axis of the septum seal 62 and housing 36. The radially inward portion of the outer lip 70 has a curved exterior which has a radius designated as Z in FIG. 9. Radius Z is preferably between 3 and 4 times that of radius Y, and more preferably is 3.5 times radius Y.

Preferably, the septum seal 62 is monolithic and unitary, and made of a flexible material. More preferably, the septum seal 62 is made of an elastomer, and most preferably is made out of polyiosoprene. An additive, such as one of lubricating additives sold by Robin Industries, Inc. Of Independence, Ohio particularly model numbers IE-131001, IE-131003, and IE-131004, may be mixed with the polyiosoprene to increase the lubricity of the seal 62. In addition, the septum seal 62 may be chlorinated using a gaseous chlorine method. Preferably the septum seal 62 chlorination level of between about 600 ppm and about 1200 ppm. Also, the material used for the septum seal 62 preferably has a durometer hardness of between 30 and 35 shore A. The thickness of walls 76, 80, and 86 is preferably on the order of 0.010 inches±0.002 inches, and the overall thicknesses of the seal range between 0.007″ and 0.035″.

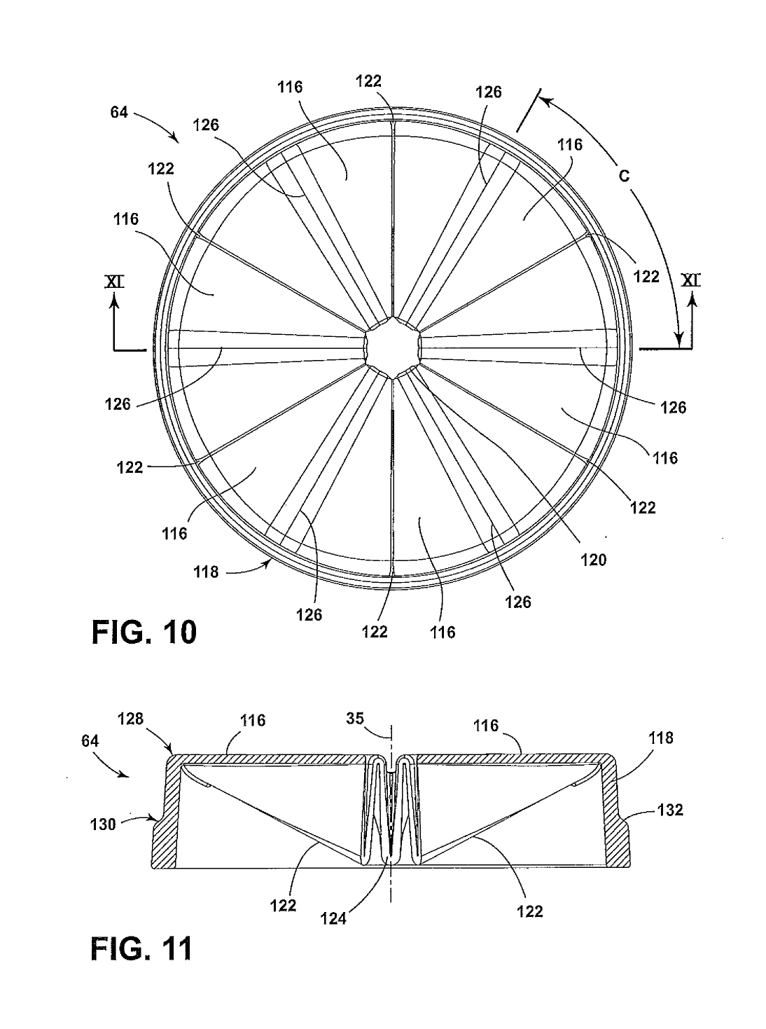

FIGS. 10-11 show the shield 64 in more detail.

The shield 64 is generally cylindrical in shape and is a unitary, monolithic structure preferably made of a polyether-based thermoplastic polyurethane. An example of such a polyurethane is Texin®, grade Rxt85A, sold by Bayer MaterialScience headquartered in Leverkusun, Germany. The material preferably has a durometer hardness of between 80 shore A and 90 shore A, and more preferably about 85 shore A.

The shield 64 has multiple indented members 116. Each indented member 116 extends radially inwardly from an outer base 118 of the shield to an inner aperture 120, which is centrally located and when in use is aligned with the longitudinal axis 35. Inner aperture 120 preferably has a width (for example, diameter) less than that of the aperture 98 of the septum seal 62, resulting in the prevention of tearing or catching of the septum seal 62 by an instrument that is inserted at an angle off of the longitudinal axis.

Each indented member 116 takes up an equal space and is equally sized and shaped as the other indented members 116. Each indented member 116 includes an indent 122 which extends from an uppermost position at the outer base 118 downwardly toward the inner aperture 120 to its lower most position. Thus, together, the indents 122 create a series of folded members 124 about the inner aperture 120. Also, due to the nature of the indents 122, a series of radially-extending ridges 126 are created, each of which defines an outer circumferential boundary of an indented member 116. The ridges 126 are each spaced an angle C from one another, and in the embodiment shown in FIG. 10, are equally spaced, making C equal to 60°.

The outer base 118 has an upper area 128 with a first outer diameter and a lower area 130 with a second outer diameter, as shown in FIG. 11. As shown, the outer diameter of the lower area 130 is greater than the outer diameter of the upper area 128, creating a shoulder 132 between the upper area 128 and the lower area 130. The respective inner diameters of the upper area 128 and the lower area 130 are the same, thus resulting in a thickness of the lower area 130 being greater than that of the upper area 128. The thickness of the lower area 130 is such that the shield 64 may be seated within the annular groove 100 of the sealing member 92 of the septum seal 62, while maintaining stability of the shield 64. The thinner upper area 128 allows for a degree of flexibility of the shield 64. The thickness of the material of the shield 64 in the area of the centrally-located folded members is between 0.007″ and 0.020″, and more preferably is about 0.014″.

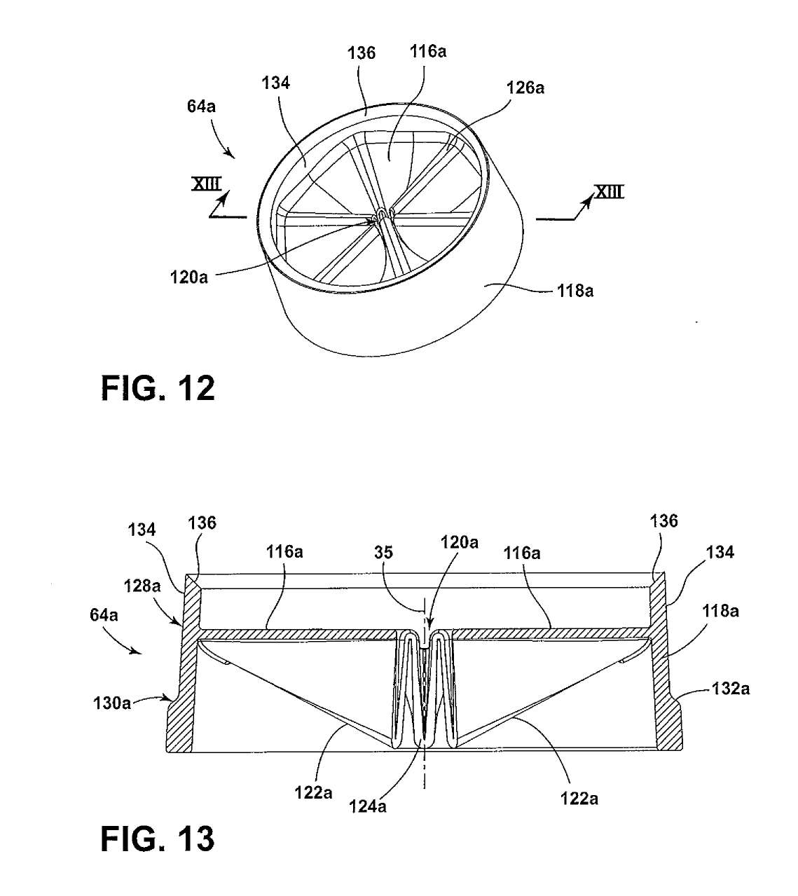

The reference numeral 64a (FIGS. 12-13) generally designates another embodiment of the shield of the present invention. Since the shield 64a is similar to the previously described shield 64, similar parts appearing in FIGS. 6 and 10-11 are represented by the same, corresponding reference number, except for the suffix “a” in the numerals of the latter. The shield 64a is essentially identical to the shield 64, except that the shield 64a has an upper area 128a which includes an upwardly extending upper wall portion 134 that extends above the inner portion of the shield 64a, generally defined by the indented members 116a and ridges 126a. The upper wall portion 134 has a tapered top 136. The shape and structure of the upper wall portion 134 provides additional protection of the septum seal 62 from aggressive instruments.

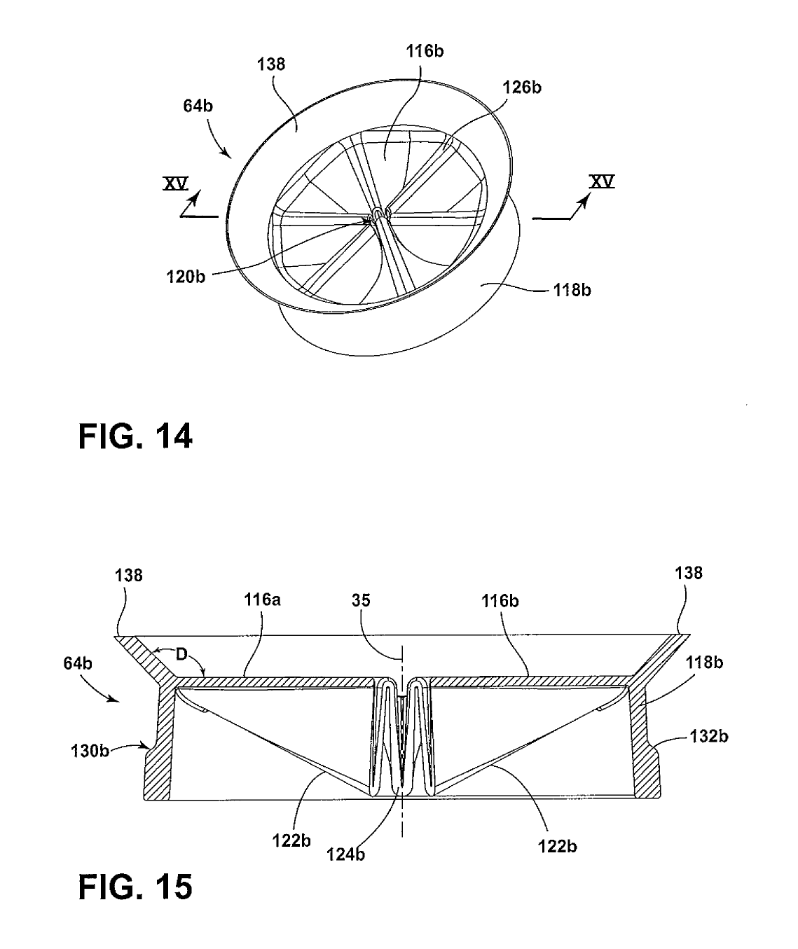

The reference numeral 64b (FIGS. 14-15) generally designates yet another embodiment of the shield of the present invention. Since the shield 64b is similar to the previously described shield 64, similar parts appearing in FIGS. 6 and 10-11 are represented by the same, corresponding reference number, except for the suffix “b” in the numerals of the latter. The shield 64b is essentially identical to shield 64, with the exception that the shield 64b includes an angled upper wall portion 138 which extends outwardly. The upper wall portion 138 increases in diameter moving upwardly, as shown and oriented in FIG. 15. The upper wall portion 138 is at an angle D with respect to the horizontal, and angle D is preferably between 40° and 50°, and more preferably 45°. The upper wall portion 138 is configured for engagement with the underside of the port 54 and acts as additional protection to the septum seal 62 from aggressive instruments.

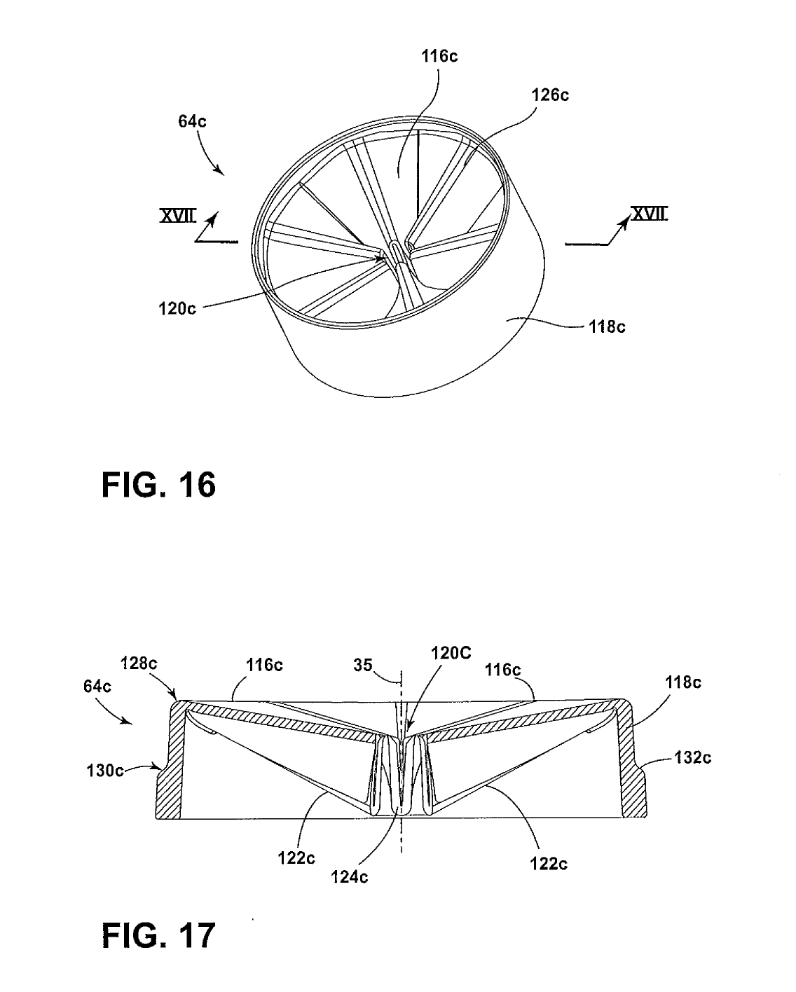

The reference numeral 64c (FIGS. 16-17) generally designates still another embodiment of the shield of the present invention. Since the shield 64c is similar to the previously described shield 64, similar parts appearing in FIGS. 6 and 10-11 are represented by the same, corresponding reference number, except for the suffix “c” in the numerals of the latter. The shield 64c is essentially identical in all respects to the shield 64, except that the indented members 116c taper toward the bottom of the shield 64c, as oriented and shown in FIG. 17, moving toward the center hole 120c. Thus, this configuration results in the top of the shield 64c having a conical or frusta-conical shape. This shield configuration acts as an extra measure to protect the septum seal 62 from aggressive instruments, while the conical profile of the shield 64c guides instruments toward the inner aperture 120c and thus toward the septum aperture 98.

In operation, the obturator assembly 14 is inserted, tip 22 first, into the port 54 of the housing 36. The tip 22 is extended through the opening 56 in the port 54, into and through the inner aperture 120 of the shield 64, into and through the aperture 98 of the septum seal 62, beyond the lower end of the housing and through the opening 49 in the lower seal 48. The tip 22 may then be extended into and through the interior 33 of the cannula 32 with the tip 22 extending beyond the distal edge of the cannula 32. The obturator assembly 14 is extended until the obturator hub 18 engages with the cap 52 of the housing 36. The obturator hub 18 is preferably aligned such that the hooks 28 engage with the slots 58 in the cap 52, temporarily affixing the obturator assembly 14 to the cannula assembly 12.

The laparoscope 16 may then be inserted into the obturator assembly 14. The laparoscope 16 is inserted through the opening 24 of the hub 18, through the hub 18, through the shaft 20, and adjacent to or into the optically clear tip 22. Once the laparoscope 16 is in the desired position, the locking mechanism 26 may be engaged to temporarily lock the laparoscope 16 in place with respect to the obturator assembly 14. Alternatively, a bladed obturator or other instrument may be inserted into and, extend out of, the cannula assembly 12.

Once the surgical procedure is complete, and/or the laparoscope 16 or obturator assembly 14 is no longer needed for the surgical procedure, the locking mechanism 26 may be disengaged, allowing the laparoscope 16 to be removed from the obturator assembly 14. The pads 30 of the hub 18 of the obturator assembly 14 may then be engaged to move the hooks 28 inwardly to allow disengagement of the hooks 28 from the slots 58 of the cap 52 of the housing 36. The obturator assembly 14 can then be removed from the cannula assembly 12.

During the surgical procedure, the stopcock valve 38 may be used to allow insufflation gas or to disallow insufflation gas into the surgical area. The lower seal 48 and septum seal 62 allow the retention of insufflation gas in the surgical site area while the obturator assembly 14 or other surgical instrument is inserted into and through the cannula assembly 12.

Even if the obturator assembly 14 or other surgical instrument is inserted into the cannula assembly 12 in a direction other than along the longitudinal axis 35, the combination of the port 54, the shield 64, and the septum seal 62 will result in adjustment of the position and/or angle of the instrument to result in the instrument being positioned substantially along the longitudinal axis 35. The structure and flexibility of the septum seal 62 and shield 64 result in an effective means by which a surgical instrument can be inserted in many different orientations into the cannula assembly 12, and directed through the interior 33 of the cannula 32 without tearing or breaking the seal assembly 60.

Although particular preferred embodiments of the invention have been disclosed in detail for illustrative purposes, it will be recognized that variations or modifications of the disclosed apparatus, including the rearrangement of parts, lie within the scope of the present invention.

What is claimed is:

1. A trocar assembly comprising:
a housing having a proximal end, at least one side wall depending from the proximal end, the proximal end and at least one side wall together defining a housing interior, and an opening adjacent or in the proximal end, the opening disposed to allow entry of an elongated instrument into the interior of the housing;
a septum seal having an outer projection attached to the housing, an outer seal wall depending from the outer projection, an inner seal wall connected to the outer seal wall and positioned inwardly with respect to the outer seal wall to define a space between the outer seal wall and the inner seal wall, and a sealing member extending inwardly from the inner seal wall, the sealing member having an aperture therein substantially aligned with the housing opening for receiving an elongated instrument therethrough, the inner seal wall and the sealing member together defining an interior seal space; and
a shield for the protection of the septum seal from being perforated by a surgical instrument, the shield residing at least partially within the interior seal space and being expandable to allow different sized instruments to pass therethrough.

2. The trocar assembly of claim 1, wherein the shield resides entirely within the interior seal space.

3. The trocar assembly of claim 1, wherein the shield is comprised of a flexible material.

4. The trocar assembly of claim 3, wherein the flexible material is a thermoplastic.

5. The trocar assembly of claim 1, and further comprising a medial seal wall connecting the outer seal wall to the inner seal wall.

6. The trocar assembly of claim 5, and further comprising a plurality of ribs each connected to both the outer seal wall and the medial seal wall.

7. The trocar assembly of claim 5, wherein the inner seal wall and the medial seal wall together define an inner ring.

8. A trocar assembly comprising:
a cannula assembly having a distal tubular portion and a proximal hub portion, the distal tubular portion extending from the proximal hub portion;
a valve at least partially disposed within the cannula proximal hub portion, the valve having a valve opening therein to allow entry of an instrument through the valve opening and into the distal tubular portion;
a seal housing attached to the cannula proximal hub portion and having a proximal opening sized to allow a surgical instrument therethrough and having at least one wall defining a seal housing interior; and
a unitary monolithic septum seal at least partially disposed within the seal housing interior and having an outer member attached to the seal housing, a support member attached to the outer member, and a sealing portion attached to the support member, the sealing portion being substantially cylindrically shaped with a first end that is open and a second end that is substantially closed by a sealing member, the sealing member having an aperture therein for receiving an instrument.

9. The trocar assembly of claim 8, the sealing portion defining an inner seal space, the trocar assembly further comprising a shield at least partially within the inner seal space.

10. The trocar assembly of claim 9, wherein the shield is cylindrical in shape.

11. The trocar assembly of claim 8, wherein the valve is a duckbill valve.

12. A universal seal assembly for a trocar, the universal seal assembly comprising:
a housing having an interior;
a seal member at least partially disposed within the interior of the housing, the seal member comprising:
a proximal lip extending annularly and engaged with the housing, the lip comprising an inner circumferential portion;
a first seal wall extending distally from the inner circumferential portion of the proximal lip and having a distal edge;
a second seal wall extending from the distal edge of the first seal wall, the first seal wall and the second seal wall together defining an outer support member;
a third seal wall extending from the second seal wall; and a sealing member extending inwardly from the third seal wall, the sealing member having an aperture therein,
the third seal wall and the sealing member together defining an interior seal space,
the first seal wall, the second seal wall, and the third seal wall positioned with respect to one another to create an s-shaped structure for allowance of off-axis movement of instruments received by the seal member.

13. The trocar assembly of claim 12, and further comprising a plurality of ribs, each rib being attached to both the first seal wall and the second seal wall.
